(19) United States
(12) Patent Application Publication
(10) Pub. No.: US 2011/0189665 A1
(43) Pub. Date: Aug. 4, 2011

(54) METHODS FOR DETECTING DRUG-RESISTANT MICROBES

(76) Inventors: Jesse D. Miller, Hudson, WI (US); Hsi-Chou C. Liu, Woodbury, MN (US); Ranjani V. Parthasarathy, Woodbury, MN (US)

(21) Appl. No.: 12/673,246

(22) PCT Filed: Aug. 12, 2008
(86) PCT No.: PCT/US08/72840

§ 371 Date: Mar. 28, 2011

Related U.S. Application Data
(60) Provisional application No. 60/964,499, filed on Aug. 13, 2007.

(51) Int. Cl. C12Q 1/68; C07H 1/06; C07H 21/04

(52) U.S. Cl. 435/6.11; 435/6.12; 536/25.4; 536/24.33

(57) ABSTRACT

The present invention provides methods and oligonucleotides for detecting drug-resistant microbes, such as vancomycin resistant Enterococcus spp., in a sample.

### CROSS REFERENCE TO RELATED APPLICATIONS

This application claims the benefit of U.S. Provisional Patent Application No. 60/964,499, filed Aug. 13, 2007, which is incorporated herein by reference.

### BACKGROUND

Members of the genus Enterococcus are gram-positive cocci that are present in nature, animals, and humans. Enterococci are part of the normal gastrointestinal and genital tract flora of humans. Of the known species, E. faecalis (80-90%) and E. faecium (5% to 10%) are most dominant in humans. Enterococci are typically not pathogenic in humans; however, they exhibit increasing levels of multidrug resistance (Kaufhold and Klein, 1995, Zentralblatt. Fuer. Bakterilogie., 282(4):507-518; Svec et al., 1996, Epidemiologie Mikrobiologie Imunologie, 45:153-157), and have been increasingly recognized as an important cause of hospital acquired infection. E. faecalis infections include urinary tract infections (UTI), bacteremia, endocarditis, and wound and abdominal-pelvic infections, accounting for 16% of all UTIs, and 8% of all becteremias.

Vancomycin resistant enterococci (VRE) have been recognized as the second most common cause of hospital infection, exceeded only by E. coli. Resistance can be chromosomally mediated (intrinsic), or plasmid or transposon mediated (acquired). VRE are characterized by resistance to virtually all available antibiotics, including vancomycin, considered the “last resort” antibiotic effective against gram-positive bacteria. Treatment options for physicians are limited, with strategies including combinations of antimicrobials or the use of new unproven compounds. Patients can be colonized and carry VRE without symptoms, with chief areas of colonization being anus, axilla, stool, perineal, umbilicus, wounds, foley catheters, and colostomy sites.

E. faecalis plasmid-born vanA, the gene which confers high level vancomycin resistance, can transfer in vitro to several gram positive microorganisms such as Staphylococcus aureus (Leclercq et al., 1989, Antimicrob. Agents Chemother. 33:10-15; Noble et al., 1992, FEMS Microbiology Letters, 72:195-198). Vancomycin resistance in clinical isolates of S. aureus, Streptococcus species, Eggerrthella lenta, and Clostridium innocuum have been reported, and the vancomycin resistance was most likely acquired from vancomycin resistant enterococci (Centers for Disease Control and Prevention. 2002. Morb. Mortal. Wkly. Rep., 51:565-567; Centers for Disease Control and Prevention. 2002, Morb. Mortal. Wkly. Rep. 51:902; Weigel et al., 2003, Science, 302:1569-1571; Weigel et al., 2007, Antimicrob. Agents Chemother., 51:231-238; Mevius et al., 1998, J. Antimicrob. Chemother., 42:275-276; Poyart et al., 1997, Antimicrob. Agents Chemother., 41:24-29; Stinear et al., 2001, Lancet, 357:855-856).

### SUMMARY OF THE INVENTION

There is a continued need for diagnostic tools directed to the early identification of drug-resistant microbes and therapeutic intervention.

The present invention includes methods for detecting a drug-resistant microbe in a biological sample. For instance, the method may include amplifying a target polynucleotide present in a biological sample to result in an amplified product, wherein the target polynucleotide is associated with resistance to vancomycin in a microbe. The amplifying may include at least one cycling step, wherein a cycling step comprises contacting the biological sample with a first primer and a second primer under suitable conditions to result in the amplification product, and contacting the amplified product with a probe under suitable conditions to hybridize the probe with the amplification product. The T_{M} of the probe may be at least 8° C. higher than the T_{M} of the first primer and the second primer. The amplified product is detected, wherein the presence of the amplified product is indicative of the presence of a drug-resistant microbe in the biological sample.

The target polynucleotide may be a vanA polynucleotide, for instance, a polynucleotide including SEQ ID NO:7, or a portion thereof. Examples of primers that can be used to amplify such a polynucleotide include, for instance, a first primer that includes a nucleotide sequence with at least about 80% identity to SEQ ID NO:1, and a second primer that includes a nucleotide sequence with at least about 80% identity to SEQ ID NO:2, wherein the primer pair amplifies a portion of SEQ ID NO:7, preferably, nucleotides 648-751 of SEQ ID NO:7. A probe useful in the methods include one with a nucleotide sequence having at least about 80% identity to SEQ ID NO:3 and/or substantially complementary to SEQ ID NO:7.

The target polynucleotide may be a vanB polynucleotide, for instance, a polynucleotide including SEQ ID NO:8, or a portion thereof. Examples of primers that can be used to amplify such a polynucleotide include, for instance, a first primer that includes a nucleotide sequence with at least about 80% identity to SEQ ID NO:4, and a second primer that includes a nucleotide sequence with at least about 80% identity to SEQ ID NO:5, wherein the primer pair amplifies a portion of SEQ ID NO:8, preferably, nucleotides 492-630 of SEQ ID NO:8. A probe useful in the methods include one with a nucleotide sequence having at least about 80% identity to SEQ ID NO:6 and/or substantially complementary to SEQ ID NO:8.

The methods may include contacting a biological sample with a probe, a first primer, and a second primer to form a mixture, wherein the primers are capable of amplifying a target polynucleotide associated with drug resistance to vancomycin in a microbe, wherein the probe will hybridize with the target polynucleotide. The T_{M} of the probe may be at least 8° C. higher than the T_{M} of the first primer and the second primer. The mixture is exposed to conditions suitable to form an amplified product if the polynucleotide associated with drug resistance is present in the biological sample. The amplified product is detected, wherein the presence of the amplified product is indicative of the presence of a drug-resistant microbe in the biological sample.

The methods may include amplifying a target polynucleotide present in a biological sample to result in an amplified product, wherein the biological sample is contacted with a first vanA primer and a second vanA primer, a first vanB primer and a second vanB primer, or a combination thereof, under suitable conditions to result in an amplified product. The first vanA primer may include a nucleotide sequence with at least about 80% identity to SEQ ID NO:1, and the second vanA primer may include a nucleotide sequence with at least about 80% identity to SEQ ID NO:2, wherein the primer pair amplifies nucleotides 648-751 of SEQ ID NO:7. The first vanB primer may include a nucleotide sequence with at least about 80% identity to SEQ ID NO:4, and the second vanB primer may include a nucleotide sequence with at least about 80% identity to SEQ ID NO:5, wherein the primer pair amplifies nucleotides 492-630 of SEQ ID NO:8. The amplified product is detected, wherein the presence of the amplified product is indicative of the presence of a drug-resistant microbe in the biological sample.

The methods may include contacting a biological sample with a first vanA primer and a second vanA primer to form a mixture, a first vanB primer and a second vanB primer to form a mixture, or a combination thereof The first vanA primer may include a nucleotide sequence with at least about 80% identity to SEQ ID NO:1, and the second vanA primer may include a nucleotide sequence with at least about 80% identity to SEQ ID NO:2, wherein the primer pair amplifies nucleotides 648-751 of SEQ ID NO:7. The first vanB primer may include a nucleotide sequence with at least about 80% identity to SEQ ID NO:4, and the second vanB primer may include a nucleotide sequence with at least about 80% identity to SEQ ID NO:5, wherein the primer pair amplifies nucleotides 492-630 of SEQ ID NO:8. The mixture is exposed to conditions suitable to form an amplified product if a vanA polynucleotide or vanB polynucleotide is present in the biological sample, and the absence of the amplified product is detected, wherein the absence of the amplified product is indicative of the absence of a drug-resistant microbe the biological sample.

In some aspects the methods may further include contacting the biological sample with a probe, wherein the T_{M} of the probe is at least 8° C. higher than the T_{M} of the first primer and the second primer. A probe may include a fluorophore and a quencher. The methods may also further include the use of a second probe, wherein the second probe has a T_{M} that is at least 8° C. higher than the T_{M} of the primers used in the method. When two probes are used, one probe may include a donor fluorophore and the second probe may include an acceptor fluorophore.

The drug-resistant microbe may be a gram positive microbe, such as, for instance, a member of the genus Staphylococcus (such as S. aureus) or the genus Enterococcus (such as E. faecalis, E. faecium, E. avium, E. gallinarum, or E. durans. The methods of the present invention may further include obtaining a biological sample. The biological sample may be from an individual suspected of infection with a drug-resistant microbe, and the biological sample may be obtained from fecal material. The detecting of the presence or absence of an amplified product may be performed after each cycling step.

The present invention also provides methods for isolating a polynucleotide. The methods may include providing a mixture of single stranded polynucleotides, exposing the mixture to an oligonucleotide under conditions suitable for specific hybridization of the oligonucleotide to a single stranded polynucleotide to result in a hybrid. The oligonucleotide includes a nucleotide sequence selected from one having at least about 80% identity to SEQ ID NO:1, at least about 80% identity to SEQ ID NO:2, at least about 80% identity to SEQ ID NO:3, at least about 80% identity to SEQ ID NO:4, at least about 80% identity to SEQ ID NO:5, or at least about 80% identity to SEQ ID NO:6. The hybrid may then be washed to remove contaminants. The oligonucleotide may include an affinity label, and the oligonucleotide may be attached to a solid phase material before or after the exposing. The mixture may be obtained from a biological sample, and the method can further include denaturing the polynucleotides present in the biological sample to result in single stranded polynucleotides.

Also included in the present invention are kits. A kit can include packaging materials, a first vanA primer, a second vanA primer, and a probe, and wherein the T_{M} of the probe is at least 8° C. higher than the T_{M} of the first and second vanA primers. The probe can include a nucleotide sequence with at least about 80% identity to SEQ ID NO:3 and hybridize to SEQ ID NO:7. The first primer may include a nucleotide sequence with at least about 80% identity to SEQ ID NO:1, and the second primer may include a nucleotide sequence with at least about 80% identity to SEQ ID NO:2, wherein the primer pair amplifies nucleotides 648-751 of SEQ ID NO:7.

A kit can include packaging materials, a first vanB primer, a second vanB primer, and a probe, and wherein the T_{M} of the probe is at least 8° C. higher than the T_{M} of the first and second vanB primers. The probe can include a nucleotide sequence with at least about 80% identity to SEQ ID NO:6 and hybridizes to SEQ ID NO:8. The first primer may include a nucleotide sequence with at least about 80% identity to SEQ ID NO:4, and the second primer may include a nucleotide sequence with at least about 80% identity to SEQ ID NO:5, wherein the primer pair amplifies nucleotides 492-630 of SEQ ID NO:8.

A probe can include a fluorophore and a quencher.

The present invention also includes isolated polynucleotides, including, for instance, a nucleotide sequence with at least about 80% identity to SEQ ID NO:1, wherein the polynucleotide amplifies a polynucleotide comprising nucleotides 648-751 of SEQ ID NO:7 when used with SEQ ID NO:2; a nucleotide sequence with at least about 80% identity to SEQ ID NO:2, wherein the polynucleotide amplifies a polynucleotide comprising nucleotides 648-751 of SEQ ID NO:7 when used with SEQ ID NO:1; a nucleotide sequence with at least about 80% identity to SEQ ID NO:4, wherein the polynucleotide amplifies a polynucleotide comprising nucleotides 492-630 of SEQ ID NO:8 when used with SEQ ID NO:5 to result in an amplified product of about 139 nucleotides; and a nucleotide sequence with at least about 80% identity to SEQ ID NO:5, wherein the polynucleotide amplifies a polynucleotide comprising nucleotides 492-630 of SEQ ID NO:8 when used with SEQ ID NO:4.

### Definitions

As used herein, the term “polynucleotide” refers to a polymeric form of nucleotides of any length, either ribonucleotides, deoxynucleotides, or peptide nucleic acids (PNA), and includes both double- and single-stranded RNA, DNA, and PNA. A polynucleotide may include nucleotide sequences having different functions, including, for instance, coding regions, and non-coding regions such as regulatory regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “oligonucleotide” refers to a polynucleotide of the present invention, typically a primer and/or a probe.

A “target polynucleotide,” as used herein, contains a polynucleotide sequence of interest, for which amplification is desired. The target sequence may be known or not known, in terms of its actual sequence.

A “coding region” is a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

“Primer,” as used herein, is an oligonucleotide that is complementary to a portion of target polynucleotide and, after hybridization to the target polynucleotide, may serve as a starting-point for an amplification reaction and the synthesis of an amplification product. A “primer pair” refers to two primers that can be used together for an amplification reaction. “vanA primers” and “vanB primers” refer to a primer pair that hybridizes to vanA or vanB polynucleotides, respectively, and can initiate amplification under the appropriate conditions. “Probe,” as used herein, is an oligonucleotide that is complementary to at least a portion of an amplification product formed using two primers. A “vanA probe” and “vanB probe” refers to a probe that hybridizes to an amplification product resulting from using vanA primers or vanB primers, respectively.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The terms “substantial complement,” “substantially complementary,” and “substantial complementarity” as used herein, refer to a polynucleotide that is capable of selectively hybridizing to a specified polynucleotide under stringent hybridization conditions. Stringent hybridization can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “specific hybridization” refers to hybridization between two polynucleotides under stringent hybridization conditions.

“Identity” refers to sequence similarity between an oligonucleotide, such as a primer or a probe, and at least a portion of a target polynucleotide or an amplification product. The similarity is determined by aligning the residues of the two polynucleotides (i.e., the nucleotide sequence of a primer or probe and a reference nucleotide sequence) to optimize the number of identical nucleotides along the lengths of their sequences; gaps in either or both sequences are permitted in making the alignment in order to optimize the number of shared nucleotides, although the nucleotides in each sequence must nonetheless remain in their proper order. The sequence similarity is typically at least about 80% identity, at least about 85% identity, at least about 90% identity, or at least about 95% identity. Sequence similarity may be determined, for example, using sequence techniques such as GCG FastA (Genetics Computer Group, Madison, Wis.), MacVector 4.5 (Kodak/IBI software package) or other suitable sequencing programs or methods known in the art. Preferably, sequence similarity between a primer and a target polynucleotide, or between a probe and an amplification product is determined using the Blastn program of the BLAST 2 search algorithm, as described by Tatusova, et al. (1999, FEMS Microbiol Lett., 174:247-250), and available through the World Wide Web, for instance at the internet site maintained by the National Center for Biotechnology Information, National Institutes of Health. Preferably, the default values for all BLAST 2 search parameters are used, including reward for match=1, penalty for mismatch=−2, open gap penalty=5, extension gap penalty=2, gap x_dropoff=50, expect=10, wordsize=11, and optionally, filter on. In the comparison of two nucleotide sequences using the BLAST search algorithm, sequence similarity is referred to as “identities.”

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a flourophore (a fluorescent quencher). This phenomenon is generally known as fluorescent resonance energy transfer (FRET). Alternatively, a quencher can dissipate the energy absorbed from a fluorophore as heat (a non-fluorescent quencher).

A “biological sample” refers to a sample obtained from eukaryotic or prokaryotic sources. Examples of eukaryotic sources include mammals, such as a human or a member of the family Muridae (a murine animal such as rat or mouse). Examples of prokaryotic sources include enterococci. The biological sample can be, for instance, in the form of a single cell, in the form of a tissue, or in the form of a fluid. Cells or tissue can be derived from in vitro culture.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, may depend upon, for example, the nature of the nucleotide sequence, temperature, and buffer conditions. These conditions may also depend on what event is desired, such as hybridization, cleavage, or strand extension.

An “isolated” polynucleotide refers to a polynucleotide that has been removed from its natural environment. A “purified” polynucleotide is one that is at least about 60% free, preferably at least about 75% free, and most preferably at least about 90% free from other components with which they are naturally associated.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

The term “and/or” means one or all of the listed elements or a combination of any two or more of the listed elements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A. Nucleotide sequence of a vanA coding region (SEQ ID NO:7).

FIG. 1B. Nucleotide sequence of a vanB coding region (SEQ ID NO:8).

### DETAILED DESCRIPTION OF ILLUSTRATIVE EMBODIMENTS

The present invention includes methods for detecting polynucleotides that are characteristic of drug-resistant prokaryotic microbes. The microbes are drug-resistant by virtue of having a vanA or vanB coding region. Preferably, the prokaryotic microbe is a member of the genus Enterococcus (referred to herein as Enterococcus ssp. or enterococci), such as, for example, E. faecalis, E. faecium, E. avium, E. gallinarum, or E. durans, more preferably, E. faecalis or E. faecium, most preferably, E. faecalis. Other examples of drug-resistant microbes include, but are not limited to, Staphylococcus spp., such as S. aureus, and Streptococcus spp. For instance, the present invention includes methods directed to detecting a portion of a vanA and/or vanB coding region present in vancomycin-resistant enterococci using amplification techniques and oligonucleotides, such as primers and probes. Using the methods of the present invention, it is possible to identify the presence of a drug-resistant microbe in a biological sample. In some aspects, the amplification techniques include the use of real-time assays. The present invention also includes the oligonucleotides described herein.

### Oligonucleotides

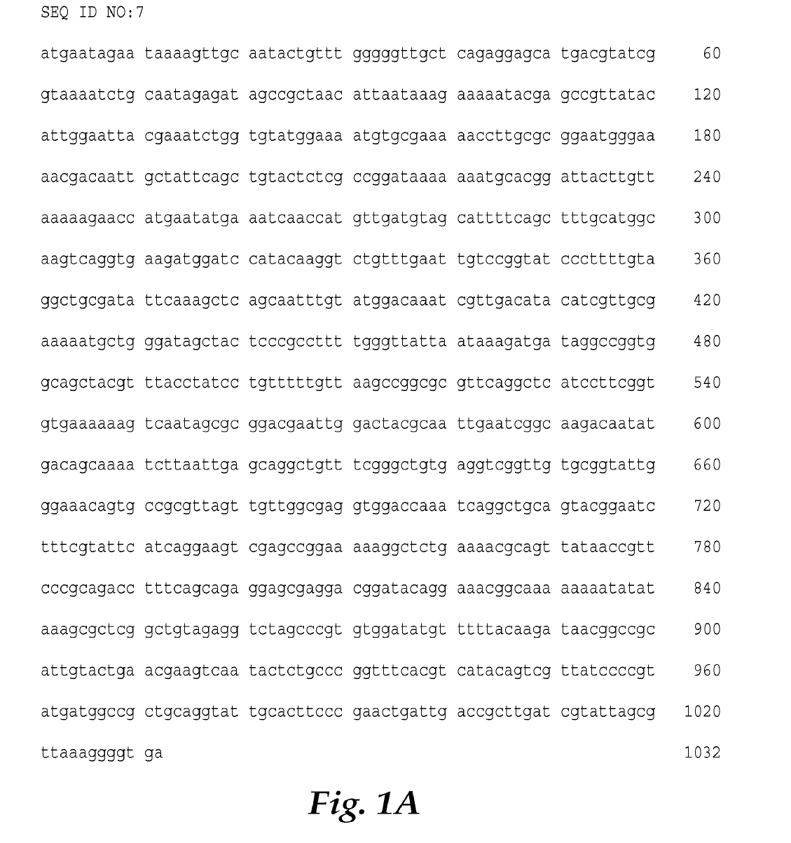

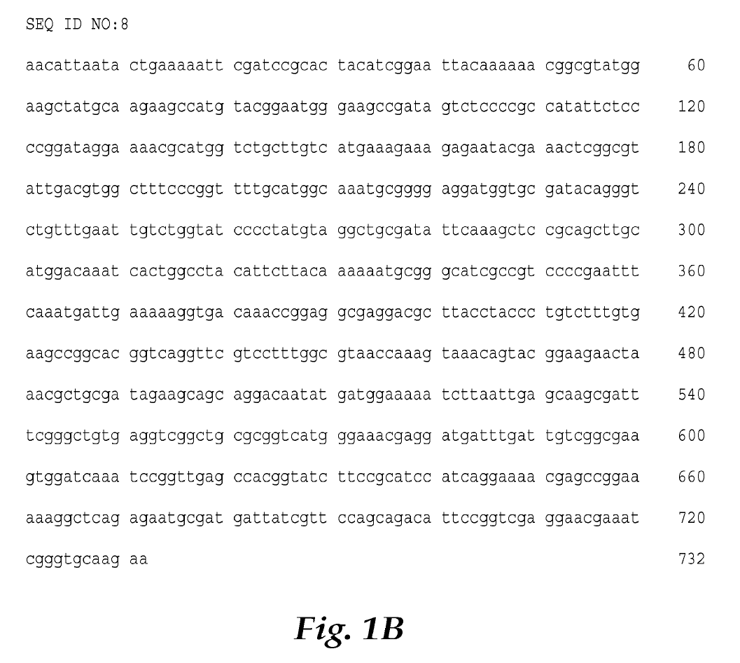

Oligonucleotides of the present invention include primers that can be used to amplify a portion of a vanA coding region. An example of a vanA coding region is disclosed at SEQ ID NO:7 (Genbank accession number AB247327, see FIG. 1A). Primers useful for amplifying a portion of a vanA coding region may amplify a region of SEQ ID NO:7, preferably a region that includes nucleotides from about 648 to about 751 of SEQ ID NO:7. Accordingly, the nucleotide sequence of a primer may correspond to nucleotides from about 648 to about 670, preferably nucleotides 648 to 670 (referred to herein as SEQ ID NO:1). Likewise, the nucleotide sequence of a primer may correspond to the complement of nucleotides from about 726 to about 751, preferably 726 to 751 (referred to herein as SEQ ID NO:2). Examples of primer pairs useful to amplify a portion of a vanA coding region include, but are not limited to, the following: SEQ ID NO:1 and SEQ ID NO:2; a primer having sequence similarity to SEQ ID NO:1 and SEQ ID NO:2; SEQ ID NO:1 and a primer having sequence similarity to SEQ ID NO:2; and a primer having sequence similarity to SEQ ID NO:1 and a primer having sequence similarity to SEQ ID NO:2.

Oligonucleotides of the present invention include primers that can be used to amplify a portion of a vanB coding region. An example of a vanB coding region is disclosed at SEQ ID NO:8 (Genbank accession number AY665551, see FIG. 1B). Primers useful for amplifying a portion of a vanB coding region may amplify a region of SEQ ID NO:8, preferably a region that includes nucleotides from about 492 to about 630 of SEQ ID NO:8. Accordingly, the nucleotide sequence of a primer may correspond to nucleotides from about 492 to about 516, preferably 492 to 516 (referred to herein as SEQ ID NO:4). Likewise, the nucleotide sequence of a primer may correspond to the complement of nucleotides from about 608 to about 630, preferably 608 to 630 (referred to herein as SEQ ID NO:5). Examples of primer pairs useful to amplify a portion of a vanA coding region include, but are not limited to, the following: SEQ ID NO:4 and SEQ ID NO:5; a primer having sequence similarity to SEQ ID NO:4 and SEQ ID NO:5; SEQ ID NO:4 and a primer having sequence similarity to SEQ ID NO:5; and a primer having sequence similarity to SEQ ID NO:4 and a primer having sequence similarity to SEQ ID NO:5.

Primers that amplify a vanA or vanB coding region can be designed using readily available computer programs, such as Primer Express® (Applied Biosystems, Foser City, Calif.), and IDT® OligoAnalyzer 3.0 (Integrated DNA Technologies, Coralville, Iowa). Factors that can be considered in designing primers include, but are not limited to, melting temperatures, primer length, size of the amplification product, and specificity. Primers useful in the amplification methods described herein typically have a melting temperature (T_{M}) that is greater than at least 56° C., at least 57° C., at least 58° C., or at least 59° C. The T_{M} of a primer can be determined by the Wallace Rule (Wallace et al., 1979, Nucleic Acids Res., 6:3543-3557) or by readily available computer programs, such as IDT Oligo Analyzer 3.0. Typically, the primers of a primer pair will have T_{M}s that vary by no greater than 4° C., no greater than 3° C., no greater than 2° C., or no greater than 1° C. Typically, two primers are long enough to hybridize to the target polynucleotide and not hybridize to other non-target polynucleotides present in microbes, preferably, enterococci, and other polynucleotides that may be present in the amplification reaction. Primer length is generally between about 15 and about 30 nucleotides (for instance, 15, 16, 18, 20, 22, 24, 26, 28, or 30 nucleotides).

A primer useful in the present invention may have sequence similarity to SEQ ID NO:1, SEQ ID NO:2, SEQ ID NO:4, or SEQ ID NO:5. Non-complementary nucleotides in such a primer with sequence similarity can be located essentially anywhere throughout the primer. In some aspects, it is preferable to preserve cytosine or guanine residues. For instance, in a primer with sequence similarity to SEQ ID NO:1, it is more preferable to alter one or more adenine or thymine residues in SEQ ID NO:1, and preserve the cytosine and guanine residues. Preferably, the first nucleotide at the 3′ end of a primer with sequence similarity is identical to the corresponding first nucleotide in SEQ ID NO:1, SEQ ID NO:2, SEQ ID NO:4, or SEQ ID NO:5.

A primer having sequence similarity to SEQ ID NO:1, SEQ ID NO:2, SEQ ID NO:4, or SEQ ID NO:5 has the activity of amplifying a target polynucleotide under the appropriate conditions. Whether such a candidate primer (i.e., a primer being compared to SEQ ID NO:1, SEQ ID NO:2, SEQ ID NO:4, or SEQ ID NO:5) having sequence similarity has the activity of amplifying a target polynucleotide can be tested using the Lightcycler® Real-Time PCR System (Roche, Indianapolis, Ind.) with the following profile: 95° C. for 30 seconds, then 45 cycles of 95° C. for 0 seconds (20° C./s slope), 60° C. for 30 seconds (20° C./s slope). Amplification can be performed in a total volume of 10 μL containing 5 microliters (μM) of sample and 5 μL of the following mixture: two primers (0.5 μL of 10 micromolar (μM) of each), MgCl₂ (2 μL of 25 mM) and LightCycler® DNA Master Hybridization Probes (1 μL of 10×, Roche). The target polynucleotide for evaluating a candidate primer having sequence similarity to either SEQ ID NO:1 or SEQ ID NO:2 is one that includes nucleotides 648 to 751 of SEQ ID NO:7. Such a nucleotide sequence is present in whole cell DNA obtained from the E. faecium designated ATCC 700221™. The target polynucleotide for evaluating a candidate primer having sequence similarity to either SEQ ID NO:4 or SEQ ID NO:5 is one that includes nucleotides 492 to 630 of SEQ ID NO:8. Such a nucleotide sequence is present in whole cell DNA obtained from the E. faecalis designated ATCC 700802™. When testing a candidate primer having sequence similarity to SEQ ID NO:1, the second primer used is SEQ ID NO:2. When testing a candidate primer having sequence similarity to SEQ ID NO:2, the second primer used is SEQ ID NO:l. When testing a candidate primer having sequence similarity to SEQ ID NO:4, the second primer used is SEQ ID NO:5. When testing a candidate primer having sequence similarity to SEQ ID NO:5, the second primer used is SEQ ID NO:4.

A primer of the present invention may further include additional nucleotides. Typically, such additional nucleotides are present at the 5′ end of the primer, and include, for instance, nucleotides that include a restriction endonuclease site, nucleotides that form a hairpin loop, and other nucleotides that permit the primer to be used as, for instance, a scorpions primer (see, for instance, Whitcombe et al., U.S. Pat. No. 6,326,145, and Whitcombe et al., 1999, Nat. Biotechnol., 17:804-817), or an amplifluor primer (see, for instance, Nazarenko et al., 1997, Nucl. Acids Res., 25:2516-2521). When a primer includes such additional nucleotides, the additional nucleotides are not included when determining if the primer has sequence similarity to SEQ ID NO:1, SEQ ID NO:2, SEQ ID NO:4, or SEQ ID NO:5. Likewise, the additional nucleotides are not included in determining the length of a primer, which is generally between about 10 and about 50 nucleotides.

Oligonucleotides of the present invention include probes that can be used to hybridize to at least a portion of an amplified product that results from the use of vanA primers or vanB primers. Such vanA probes useful herein hybridize to a region that includes nucleotides from about 671 to about 725 of SEQ ID NO:7, preferably nucleotides 684 to 712 of SEQ ID NO:7. Such vanB probes useful herein hybridize to a region that includes nucleotides from about 517 to about 607 of SEQ ID NO:8, preferably nucleotides 549 to 573 of SEQ ID NO:8.

Typically, a vanA probe is designed to be used in a method of the present invention with a particular set of vanA primers, and a vanB probe is designed to be used in a method of the present invention with a particular set of vanB primers. Designing vanA and vanB probes can be done in a manner similar to designing the primers described herein. Factors that can be considered in designing probes useful in the methods described herein include, but are not limited to, melting temperature, length, location of the probe with respect to the primers. Typically, a probe will have a T_{M} that is greater than the highest T_{M} of the primers with which the probe is to be used. Preferably, a probe has a T_{M} that is at least about 8° C. greater, at least about 8.5° C. greater, at least about 9° C. greater, at least about 9.5° C. greater, or at least about 10° C. greater than the highest T_{M} of the primer pair with which the probe is to be used. Typically, the greater Tm permits the probe to hybridize before the primer, which aids in maximizing the labeling of each amplification product with probe.

Typically, a probe is long enough to hybridize to the target polynucleotide (and the amplification product) and not hybridize to other non-target polynucleotides present in a microbe, preferably, an enterococci, and other polynucleotides that may be present in the amplification reaction. Probe lengths are generally between about 15 nucleotides and about 30 nucleotides. Preferably, a probe and the primers with which the probe is used will not hybridize to the same nucleotides of an amplification product. A probe will hybridize to one strand of an amplified product, and is typically designed to hybridize to the amplified product before the primer that hybridizes to that strand. In some aspects of the present invention, a probe hybridizes to one strand of an amplified product within no more than 1, 2, 3, 4, or 5 nucleotides of the primer that hybridizes to the same strand. In some aspects of the invention that involve the use of two probes, the two probes preferably hybridize to the same strand of an amplified product, and the two probes may optionally hybridize to the same amplification product within 1, 2, 3, 4, or 5 nucleotides of each other.

A probe useful in the present invention may have sequence similarity to SEQ ID NO:3 or SEQ ID NO:6. Non-complementary nucleotides in such a probe with sequence similarity can be located essentially anywhere throughout the probe. In some aspects, it is preferable to preserve cytosine or guanine residues. A probe having sequence similarity to SEQ ID NO:3 or SEQ ID NO:6 has the activity of hybridizing to an amplified product under the same conditions the primers of a primer pair will hybridize. Whether such a candidate probe (i.e., a probe being compared to SEQ ID NO:3 or SEQ ID NO:6) having sequence similarity has this activity can be tested by including a candidate probe in an amplification reaction with a primer pair, and determining whether the candidate probe forms a hybrid with the amplification product during the annealing step. The target polynucleotide for evaluating a candidate probe having sequence similarity to SEQ ID NO:3 is one that includes nucleotides 648 to 751 of SEQ ID NO:7, and the target polynucleotide for evaluating a candidate probe having sequence similarity to SEQ ID NO:6 is one that includes nucleotides 492 to 630 of SEQ ID NO:8. When testing a candidate probe having sequence similarity to SEQ ID NO:3, SEQ ID NO:1 and SEQ ID NO:2 are used as the primer pair. When testing a candidate probe having sequence similarity to SEQ ID NO:6, SEQ ID NO:4 and SEQ ID NO:5 are used as the primer pair.

A probe of the present invention may further include additional nucleotides. Such additional nucleotides may be present at either the 5′ end, the 3′ end, or both, and include, for instance, nucleotides that form a hairpin loop, and other nucleotides that permit the probe to be used as, for instance, a molecular beacon. When a probe includes such additional nucleotides, the additional nucleotides are not included when determining if the probe has sequence similarity to SEQ ID NO:7 or SEQ ID NO:8. Likewise, the additional nucleotides are not included when determining the length of a probe, which is generally between about 15 and about 30 nucleotides.

Nucleotides of an oligonucleotide of the present invention may be modified. Such modifications can be useful to increase stability of the polynucleotide in certain environments. Modifications can include a nucleic acid backbone, base, sugar, or any combination thereof. The modifications can be synthetic, naturally occurring, or non-naturally occurring. A polynucleotide of the present invention can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide nucleic acids (Nielson et al., U.S. Pat. No. 5,539,082; Egholm et al., Nature, 1993, 365:566-568). Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides.

Oligonucleotides may include a label. Exemplary labels include, but are not limited to, fluorophore labels (including, e.g., quenchers or absorbers), non-fluorescent labels, colorimetric labels, chemiluminescent labels, bioluminescent labels, radioactive labels, mass-modifying groups, affinity labels, magnetic particles, antigens, enzymes (including, e.g., peroxidase, phosphatase), substrates, and the like. Labels may provide signals detectable by fluorescence, radioactivity, colorimetry, gravimetry, X-ray diffraction or absorption, magnetism, enzymatic activity, and the like. Affinity labels provide for a specific interaction with another molecule. Examples of affinity labels include, for instance, biotin, avidin, streptavidin, dinitrophenyl, digoxigenin, cholesterol, polyethyleneoxy, haptens, and peptides such as antibodies.

In certain aspects a label is a fluorophore. Fluorophore labels include, but are not limited to, dyes of the fluorescein family, the carboxyrhodamine family, the cyanine family, and the rhodamine family. Other families of dyes that can be used in the invention include, e.g., polyhalofluorescein-family dyes, hexachlorofluorescein-family dyes, coumarin-family dyes, oxazine-family dyes, thiazine-family dyes, squaraine-family dyes, chelated lanthanide-family dyes, the family of dyes available under the trade designation Alexa FluorJ, from Molecular Probes, and the family of dyes available under the trade designation BodipyJ, from Invitrogen (Carlsbad, Calif.). Dyes of the fluorescein family include, e.g., 6-carboxyfluorescein (FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), 2′-chloro-7′-phenyl-1,4-dichloro-6-carboxyfluorescein (VIC), 6-carboxy-X-rhodamine (ROX), and 2′,4′,5′,7′-tetrachloro-5-carboxy-fluorescein (ZOE). Dyes of the carboxyrhodamine family include tetramethyl-6-carboxyrhodamine (TAMRA), tetrapropano-6-carboxyrhodamine (ROX), Texas Red, R110, and R6G. Dyes of the cyanine family include Cy2, Cy3, Cy3.5, Cy5, Cy5.5, and Cy7. Fluorophores are readily available commercially from, for instance, Perkin-Elmer (Foster City, Calif.), Molecular Probes, Inc. (Eugene, Oreg.), and Amersham GE Healthcare (Piscataway, N.J.).

The label may be a quencher. Quenchers may be fluorescent quenchers or non-fluorescent quenchers. Fluorescent quenchers include, but are not limited to, TAMRA, ROX, DABCYL, DABSYL, cyanine dyes including nitrothiazole blue (NTB), anthraquinone, malachite green, nitrothiazole,and nitroimidazole compounds. Exemplary non-fluorescent quenchers that dissipate energy absorbed from a fluorophore include those available under the trade designation Black HoleJ, from Biosearch Technologies, Inc. (Novato, Calif.), those available under the trade designation Eclipse DarkJ, from Epoch Biosciences (Bothell, Wash.), those available under the trade designation QxlJ, from Anaspec, Inc. (San Jose, Calif.), and those available under the trade designation Iowa BlackJ, from Integrated DNA Technologies (Coralville, Iowa).

Typically, a fluorophore and a quencher are used together, and may be on the same or different oligonucleotides. When paired together, a fluorophore and fluorescent quencher can be referred to as a donor fluorophore and acceptor fluorophore, respectively. A number of convenient fluorophore/quencher pairs are known in the art (see, for example, Glazer et al, Current Opinion in Biotechnology, 1997; 8:94-102; Tyagi et al., 1998, Nat. Biotechnol., 16:49-53) and are readily available commercially from, for instance, Molecular Probes (Junction City, Oreg.), and Applied Biosystems (Foster City, Calif.). Examples of donor fluorophores that can be used with various acceptor fluorophores include, but are not limited to, fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2-,2′-disulfonic acid derivatives. Acceptor fluorophores typically depend upon the donor fluorophore used. Examples of acceptor fluorophores include, but are not limited to, LC J-Red 640, LC J-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorophores are readily available commercially from, for instance, Molecular Probes or Sigma Chemical Co. (St. Louis, Mo.).

Examples of probes useful in real-time assays using donor and acceptor fluorophores include, but are not limited to, adjacent probes (Cardullo et al., 1988, Proc. Natl. Acad. Sci. USA, 85:8790-8794; Wittwer, 1997, BioTechniques, 22:130-131), and Taqman probes (Holland et al., 1991, Proc. Natl. Acad. Sci. USA, 88:7276-7280; Livak et al., 1995, PCR Methods Appl., 4:357-62). Examples of probes and primers useful in real-time assays using fluorphores and non-fluorescent quenchers include, but are not limited to, molecular beacons (Tyagi et al., 1996, Nat. Biotechnol., 14:303-308; Johansson et al., 2002, J. Am. Chem. Soc., 124:6950-6956), scorpion primers (including duplex scorpion primers) (Whitcombe et al., U.S. Pat. No. 6,326,145; Whitcombe et al., 1999, Nat. Biotechnol., 17:804-817), amplifluor primers (Nazarenko et al., 1997, Nucl. Acids res., 25:2516-2521), and light-up probes (Svanvik et al., 2000, Anal. Biochem., 287:179-182).

A polynucleotide of the present invention can be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual, Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors and viral vectors. Examples of viral vectors include, for instance, adenoviral vectors, adeno-associated viral vectors, lentiviral vectors, retroviral vectors, and herpes virus vectors. Typically, a vector is capable of replication in a bacterial host, for instance E. coli. Preferably the vector is a plasmid. Vectors may also include a vanA coding region, such as SEQ ID NO:7, or a portion thereof, preferably nucleotides from about 648 to about 751 of SEQ ID NO:7, or a vanB coding region, such as SEQ ID NO:8, or a portion thereof, preferably nucleotides from about 492 to about 630 of SEQ ID NO:8. Such vectors can be used as, for instance, control target polynucleotides.

Selection of a vector depends upon a variety of desired characteristics in the resulting construct, such as a selection marker, vector replication rate, and the like. Suitable host cells for cloning or expressing the vectors herein are prokaryotic cells. Suitable prokaryotic cells include eubacteria, such as gram-negative microbes, for example, E. coli. Vectors can be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells.

Polynucleotides of the present invention can be produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide then isolated from the cell.

Polynucleotides which are identical or sufficiently identical to a nucleotide sequence contained in one of SEQ ID NO:1, SEQ ID NO:2, SEQ ID NO:3, or SEQ ID NO:4, or fragments thereof, may be used as primers for a nucleic acid amplification (PCR) reaction to detect target polynucleotides that are characteristic of drug-resistant prokaryotic microbes of the genus Enterococcus (and genes encoding homologs and orthologs from microbes belonging to genera other than Enterococcus that have a high sequence similarity to the target polynucleotide sequence). Typically these primer polynucleotides are from at least about 80% identical to at least about 95% identical (e.g., having at least about 80% sequence identity, at least about 85% sequence identity, at least about 90% sequence identity, or at least about 95% sequence identity) to one of the nucleotide sequences set forth in SEQ ID NO:1, SEQ ID NO:2, SEQ ID NO:3, or SEQ ID NO:4.

Polynucleotides which are identical or sufficiently identical to a nucleotide sequence contained in one of SEQ ID NO:3 or SEQ ID NO:6, or fragments thereof, may be used as probes for a nucleic acid detection reaction (e.g., hybridization) to detect target polynucleotides that are characteristic of drug-resistant prokaryotic microbes of the genus Enterococcus (and genes encoding homologs and orthologs from microbes belonging to genera other than Enterococcus that have a high sequence similarity to the target polynucleotide sequence). Typically these probe polynucleotides are from at least about 80% identical to at least about 95% identical (e.g., having at least about 80% sequence identity, at least about 85% sequence identity, at least about 90% sequence identity, or at least about 95% sequence identity) to one of the nucleotide sequences set forth in SEQ ID NO:3 or SEQ ID NO:6.

### Methods of Use

The present invention includes methods for detecting polynucleotides that are characteristic of drug-resistant prokaryotic microbes, preferably, a member of the genus Enterococcus, such as, for example, E. faecalis, E. faecium, E. avium, E. gallinarum, or E. durans, more preferably, E. faecalis or E. faecium, most preferably, E. faecalis. Other examples of drug-resistant microbes include, but are not limited to, Staphylococcus spp., such as S. aureus, and Streptococcus spp. If the sample is obtained from a subject, the method may be used to determine whether the subject is infected with the drug-resistant microbe. The methods of this aspect of the present invention typically include contacting a target polynucleotide with a primer pair of the present invention, amplifying the polynucleotide, and detecting the resulting amplified product.

The target polynucleotide used in the methods may be present in a sample. The sample can be a food sample, a beverage sample, a fermentation broth, a forensic sample, an environmental sample (e.g., soil, dirt, garbage, sewage, or water), or a biological sample. Preferably, the sample is a biological sample. A “biological sample” refers to a sample obtained from eukaryotic or prokaryotic sources. Examples of eukaryotic sources include mammals, such as a human or a member of the family Muridae (a murine animal such as rat or mouse). Examples of prokaryotic sources include enterococci, and other microbes containing an endogenous or recombinant vanA or vanB coding region.

The biological sample can be, for instance, in the form of a single cell, in the form of a tissue, or in the form of a fluid. Cells or tissue can be derived from in vitro culture. When obtained from an animal, the biological sample can be obtained from, for instance, anal swabs, perirectal swabs, stool samples, blood, and/or body fluids. In some aspects, the biological sample is obtained from a subject suspected of having an enterococci infection. A sample may be an isolated polynucleotide, for instance, a polynucleotide present in a vector as described herein, or an polynucleotide isolated using methods described hereinbelow.

The sample can be a solid sample (e.g., solid tissue) that is dissolved or dispersed in water or an organic medium, or from which the polynucleotide has been extracted into water or an organic medium. For example, the sample can be an organ homogenate. Thus, the sample can include previously extracted polynucleotides.

In some aspects, the sample may be incubated with an enrichment broth to enrich for microbes, preferably, enterococci, that are present. The sensitivity of a sample for such a microbe can be enhanced by including an enrichment culture process prior to sample preparation to extract the polynucleotides for amplification and detection. Sample material (e.g., a biological sample) is used to inoculate a suitable medium/broth supplemented with the antibiotic(s) at a certain concentration which kills other microbes in the sample but allows for proliferation of the antibiotic-resistant microbe, and then the culture is incubated at a suitable temperature (e.g., 37° C.) for a period of time (for instance, between 18 and 24 hours). Preferably, the antibiotic is vancomycin, which may be used at a concentration, for instance, of between 4 milligrams/milliliter (mg/ml) and 8 mg/ml. At the end of the enrichment culture process, the sample with the microbe of interest is collected from a portion of the culture by centrifugation, filtration, or other suitable methods, and then used in methods of the present invention involving amplification and detection.

The polynucleotides may be from an impure, partially pure, or a pure sample. The purity of the original sample is not critical, as polynucleotides may be obtained from even grossly impure samples. For example, polynucleotides may be obtained from an impure sample of a biological fluid such as blood, saliva, feces, or tissue. If a sample of higher purity is desired, the sample may be treated according to any conventional means known to those of skill in the art prior to undergoing the methods of the present invention. A polynucleotide may be isolated using methods described hereinbelow.

Complex biological samples (feces, blood, food, tissue, sputum, etc.) may contain solid debris and/or amplification inhibitors. Solid debris is commonly removed by sedimentation or centrifugation (separate supernatant from solids), filtration, etc. Amplification inhibitors are often removed by treatment with protein denaturants or proteases, dilution, etc. Undesired polynucleotide-containing cells may be reduced by selective lysis, differential centrifugation, filtration, etc.

Specific microbes, preferably, enterococci, may be removed from a sample prior to amplification of a target polynucleotide present in an Enterococcus ssp. For example, a biological sample can be exposed to a matrix functionalized with an agent that will interact with enterococci, but not interact with other components present in a biological sample. The interaction is a reversible retention via a wide variety of mechanisms, including weak forces such as Van der Waals interactions, electrostatic interactions, affinity binding, or physical trapping. Examples of useful agents include, but are not limited to, specific interactions, such as those mediated by an anti-enterococci antibody, and non-specific interactions. Examples of agents that can be used to mediate non-specific interactions with microbes include silica, zirconia, alumina beads, metal colloids such as gold, and gold coated sheets that have been functionalized through mercapto chemistry, for example (Parthasarathy, U.S. Provisional Application Ser. No. 60/913,813, filed Apr. 25, 2007, Attorney Docket No. 62470US002).

Agents that interact with enterococci can be present on any solid phase material. Examples include polyolefin, polystyrene, nylon, poly(meth)acrylate, polyacrylamide, polysaccharide, and fluorinated polymers, as well as resins such as agarose, latex, cellulose, and dextran. The solid material may be in any form, preferably in the form of particulate material (e.g., particles, beads, microbeads, microspheres) or any other form (e.g., fibrils) that can be introduced into a microfluidic device (Parthasarathy, U.S. Provisional Application Ser. No. 60/913,813, filed Apr. 25, 2007, Attorney Docket No. 62470US002).

Prior to use in an amplification reaction, polynucleotides present in a sample, such as a biological sample, may be prepared for amplification. Treatments for preparing polynucleotides for amplification arc well known in the art and used routinely. Polynucleotides can be extracted from a biological sample. Extraction typically includes lysis of microbes to release polynucleotides. Lysis herein is the physical disruption of the membranes of the cells. Extraction can be accomplished by the use of standard techniques and reagents. Examples include, for instance, boiling, hydrolysis with proteinases, exposure to ultrasonic waves, detergents, strong bases, or organic solvents such as phenol chloroform (Lin et al., U.S. Pat. No. 5,620,852; Kellogg et al., U.S. Pat. No. 5,010,183). Polynucleotides can be prepared by use of particles, such as magnetic glass particles, under conditions to bind the polynucleotides, followed by washing to remove impurities, and then obtaining purified polynucleotides with a wash designed to remove the bound polynucleotides (MagNA Pure, International Publication No. WO 01/37291 A1).

The polynucleotides used as targets in the methods of the present invention may be of any molecular weight and in single-stranded form, double-stranded form, circular, plasmid, etc. Various types of polynucleotides can be separated from each other (e.g., RNA from DNA, or double-stranded DNA from single-stranded DNA). For example, polynucleotides of at least about 100 bases in length, longer molecules of 1,000 bases to 10,000 bases in length, and even high molecular weight nucleic acids of up to about 3.2 megabases can be used in the methods of the present invention.

Polynucleotide amplification, such as the polymerase chain reaction (PCR), is a method for the enzymatic amplification of specific segments of polynucleotides. The amplification is based on repeated cycles of the following basic steps: denaturation of double-stranded polynucleotides, followed by primer annealing to the target polynucleotide, and primer extension by a polymerase (Mullis et al., U.S. Pat. No. 4,683,195, Mullis, U.S. Pat. No. 4,683,202, and Mullis et al., U.S. Pat. No. 4,800,159). The primers are designed to anneal to opposite strands of the DNA, and are positioned so that the polymerase-catalyzed extension product of one primer can serve as the template strand for the other primer. The amplification process can result in the exponential increase of discrete polynucleotide fragments whose length is defined by the 5′ ends of the primers.

Generally, these steps are achieved in a cycling step. A typical cycling step used in DNA amplification involves two target temperatures to result in denaturation, annealing, and extension. The first temperature is an increase to a predetermined target denaturation temperature high enough to separate the double-stranded target polynucleotide into single strands. Generally, the target denaturation temperature of a cycling step is approximately 92° C. to 98° C., such as 94° C. to 96° C., and the reaction is held at this temperature for a time period ranging between 0 seconds to 5 minutes. The temperature of the reaction mixture is then lowered to a second target temperature. This second target temperature allows the primers (and probe(s), if present) to anneal or hybridize to the single strands of DNA, and promote the synthesis of extension products by a DNA polymerase. Generally, the second temperature of a cycling step is approximately 57° C. to 63° C., such as 59° C. to 61° C., and the reaction is held at this temperature for a time period ranging between 0 seconds to 1 minute. This second temperature can vary greatly depending upon the primers (and probe(s), if present) and target polynucleotide used. This completes one cycling step. The next cycle then starts by raising the temperature of the reaction mixture to the denaturation temperature. Typically, the cycle is repeated to provide the desired result, which may be to produce a quantity of DNA and/or detect an amplified product. For use in detection, the number of cycling steps will depend on the nature of the sample. For instance, if the sample is a complex mixture of polynucleotides, more cycling steps may be required to amplify the target polynucleotide sufficient for detection. Generally, the cycling steps are repeated at least about 20 times, but may be repeated as many as 40, 60, or even 100 times. As will be understood by the skilled artisan, the above description of the thermal cycling reaction is provided for illustration only, and accordingly, the temperatures, times and cycle number can vary depending upon the nature of the thermal cycling reaction and application.

Optionally, a third temperature is also used in a cycling step. The use of three target temperatures also results in denaturation, annealing, and extension, but separate target temperatures are used for the denaturation, annealing, and extension. When three target temperatures are used the annealing temperatures generally range from 45° C. to 60° C., depending upon the application. The third target temperature is for extension, is typically held for a time period ranging between 30 seconds to 10 minutes, and occurs at a temperature range between the annealing and denaturing temperatures.

DNA polymerases for use in the methods and compositions of the present invention are capable of effecting extension of a primer according to the methods of the present invention. Accordingly, a preferred polymerase is one that is capable of extending a primer along a target polynucleotide. Preferably, a polymerase is thermostable. A thermostable polymerase is a polymerase that is heat stable, i.e., the polymerase catalyzes the formation of primer extension products complementary to a template and does not irreversibly denature when subjected to the elevated temperatures for the time necessary to effect denaturation of double-stranded template nucleic acids. Useful thermostable polymerases are well known and used routinely. Thermostable polymerases have been isolated from Thermus flavus, T. ruber, T. thermophilus, T. aquaticus, T. lacteus, T. rubens, Bacillus stearothermophilus, and Methanothermus fervidus.

A polymerase typically initiates synthesis at the 3′-end of a primer annealed to a target polynucleotide, and proceeds in the 5′-direction along the target polynucleotide. A polymerase may possess a 5′ to 3′ exonuclease activity, and hydrolyze intervening, annealed probe(s), if present, to release portions of the probe(s), until synthesis terminates. Examples of suitable polymerases having a 5′ to 3′ exonuclease activity include, for example, Tfi, Taq, and FastStart Taq (Roche). In other aspects, the polymerase has little or no 5′ to 3′ exonuclease activity so as to minimize degradation of primer, termination or primer extension polynucleotides. This exonuclease activity may be dependent on factors such as pH, salt concentration, whether the target is double stranded or single stranded, and so forth, all of which are familiar to one skilled in the art. Examples of suitable polymerases having little or no 5′ to 3′ exonuclease activity include Klentaq (Sigma, St. Louis, Mo.).

Typically, amplification involves mixing one or more target polynucleotides which can have different sequences with a “master mix” containing the reaction components for performing the amplification reaction and subjecting this reaction mixture to temperature conditions that allow for the amplification of the target polynucleotide. The reaction components in the master mix can include a buffer which regulates the pH of the reaction mixture, magnesium ion, one or more of the natural nucleotides (corresponding to adenine, cytosine, guanine, and thymine or uracil, often present in equal concentrations), that provide the energy and nucleosides necessary for the synthesis of an amplification product, primer pairs that bind to the target in order to facilitate the initiation of polynucleotide synthesis, a polymerase that adds the nucleotides to the complementary strand being synthesized, and optionally, one or more probes. One skilled in the art will recognize that a successful amplification reaction will not occur in the absence of a target polynucleotide, although the presence of a target polynucleotide is not required to perform the present methods.

The presence or absence of an amplified product can be determined or its amount measured. Detecting an amplified product can be conducted by standard methods well known in the art and used routinely. The detecting may occur, for instance, after multiple amplification cycles have been run, or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. The most convenient label is typically fluorescent, which may be used in various formats including, but are not limited to, the use of donor fluorophore labels, acceptor fluorophore labels, flourophores, quenchers, and combinations thereof The types of assays using the various formats may include the use of one or more primers that arc labeled (for instance, scorpions primers, amplifluor primers), one or more probes that are labeled (for instance, adjacent probes, Taqman probes, light-up probes, molecular beacons), or a combination thereof. The skilled person will understand that in addition to these known formats, new types of formats are routinely disclosed. The present invention is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction.

As an alternative to detection using a labeled primer and/or probe, an amplification product can be detected using a polynucleotide binding dye such as a fluorescent DNA binding dye. Examples include, for instance, SYBRGreen or SYBRGold (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

Controls can be included when an amplification reaction is run. Control target polynucleotides can be amplified from a positive control sample (e.g., a target polynucleotide other than vanA or vanB) using, for example, control primers and control probes. Positive control samples can also be used to amplify a target vanA or vanB polynucleotide. Such a control can be amplified internally (e.g., within each amplification reaction) or in separate samples run side-by-side with a subject's sample. Each run may also include a negative control that, for example, lacks a target vanA or vanB polynucleotide.

It is understood that the present invention is not limited by the device used to conduct the amplification and detection of the amplified product. For example, suitable devices may include conventional amplification devices such as, for instance, the Lightcycler® Real-Time PCR System (Roche) (University of Utah Research Foundation, International Publication Nos. WO 97/46707, WO 97/46714, and WO 97/46712), MX3005p (Stratagene, La Jolla, Calif.), and amplification devices available from Bio-Rad. It may be preferred that the present invention is practiced in connection with a microfluidic device. “Microfluidic” refers to a device with one or more fluid passages, chambers, or conduits that have at least one internal cross-sectional dimension, e.g., depth, width, length, diameter, etc., that is less than 500 μm, and typically between 0.1 μm and 500 μm. Typically, a microfluidic device includes a plurality of chambers (e.g., amplification reaction chambers, loading chambers, and the like), each of the chambers defining a volume for containing a sample. Some examples of potentially suitable microfluidic devices are described in U.S. Patent Application Publication Nos. 2002/0064885 (Bedingham et al.); US2002/0048533 (Bedingham et al.); US2002/0047003 (Bedingham et al.); and US2003/138779 (Parthasarathy et al.); as well as U.S. Pat. No. 6,627,159 (Bedingham et al.); U.S. Pat. No. 6,720,187 (Bedingham et al.); U.S. Pat. No. 6,734,401 (Bedingham et al.); U.S. Pat. No. 6,814,935 (Harms et al.); U.S. Pat. No. 6,987,253 (Bedingham et al.); U.S. Pat. No. 7,026,168 (Bedingham et al.); and U.S. Pat. No. 7,164,107 (Bedingham et al.).

The present invention also includes methods for isolating, preferably, purifying a polynucleotide. The methods of this aspect of the present invention typically include providing a mixture that contains single stranded polynucleotides, exposing the mixture to an oligonucleotide of the present invention under suitable conditions for specific hybridization of the oligonucleotide to a single stranded polynucleotide to result in a hybrid, and isolating the hybrid from non-hybridized single stranded polynucleotides. Such methods may be used to prepare a sample prior to amplification of a target polynucleotide present in a drug-resistant enterococci.

The mixture may be obtained from a sample, preferably, a biological sample. Typically, the sample may contain a drug-resistant microbe, preferably, an enterococci. The sample may be prepared for isolation by extraction as described hereinabove. The polynucleotides in the mixture may be impure (e.g., other cellular materials and/or solid debris are present), partially pure, or purified. The polynucleotides in the mixture may be denatured using well known and routine methods. Examples of such methods include, for instance, heating, or exposure to alkaline conditions.

The mixture of single stranded polynucleotides is exposed to an oligonucleotide of the present invention in suitable conditions for specific hybridization of the oligonucleotide and the complementary single stranded polynucleotide. The oligonucleotide typically includes a label, preferably an affinity label. Conventional hybridization formats which are particularly useful include those where oligonucleotide is immobilized on a solid support (solid-phase hybridization) and those where the polynucleotides, (both single stranded polynucleotides and oligonucleotides) are all in solution (solution hybridization).

In solid-phase hybridization formats, the oligonucleotide is typically attached to a solid phase material prior to the hybridization. In solution hybridization formats, the oligonucleotide is typically attached to a solid phase material after the hybridization. In both formats, the attachment is mediated by a label, preferably an affinity label, that is attached to the oligonucleotide. Examples of useful solid phase materials include, for instance, polyolefin, polystyrene, nylon, poly(meth)acrylate, polyacrylamide, polysaccharide, and fluorinated polymers, as well as resins such as agarose, latex, cellulose, and dextran. The solid material may be in any form, preferably in the form of particulate material (e.g., particles, beads, microbeads, microspheres) or any other form (e.g., fibrils) that can be introduced into a microfluidic device (Parthasarathy, U.S. Provisional Application Ser. No. 60/913,813, filed Apr. 25, 2007, Attorney Docket No. 62470US002).

The hybridization is performed under suitable conditions for selectively binding the labeled oligonucleotide to the substantially complementary, preferably complementary, single stranded polynucleotides present in the mixture, e.g., stringent hybridization conditions. General methods for hybridization reactions and probe synthesis arc disclosed in Molecular Cloning by T. Maniatis, E. F. Fritsch and J. Sambrook, Cold Spring Harbor Laboratory, 1982. Preferably, the hybridization conditions include the use of a hybridization buffer such as 6×SSC, 5× Denhardt's reagent, 0.5% (w/v) SDS, and a blocking reagent such as 100 μg/ml salmon sperm. Hybridization may be allowed to occur at 68° C. for at least 2 hours. After the hybridization, (and attachment of the labeled oligonucleotide, if appropriate), the non-hybridized polynucleotides, and any other materials that may be present, can be removed by washing at room temperature several times in a solution containing 2×SSC and 0.5% SDS. Optionally, the isolated polynucleotide may be purified by denaturing the hybrid to release the isolated polypeptide and removing the bound oligonucleotide and solid support.

### Kits

The present invention provides kits, which can include oligonucleotides of the present invention, such as, for instance, a primer pair, and optionally, a probe. Other components that can be included within kits of the present invention include conventional reagents such as a master mix, solid phase support(s), hybridization solutions, external positive or negative controls, and the like.

The kits typically include packaging material, which refers to one or more physical structures used to house the contents of the kit. The packaging material can be constructed by well-known methods, preferably to provide a sterile, contaminant-free environment. The packaging material may have a marking that indicates the contents of the kit. In addition, the kit contains instructions indicating how the materials within the kit are employed. As used herein, the term “package” refers to a solid matrix or material such as glass, plastic, paper, foil, and the like.

“Instructions” typically include a tangible expression describing the various methods of the present invention, including sample preparation conditions, amplification conditions, and the like.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Detection of vanA and vanB Genes in Nucleic Acid Samples using Gene-Specific Primers and Probes

A nucleic-acid based detection strategy to identify glycopeptide-resistance genes may be useful in assays to discern whether a sample contains microbes that are able to survive treatment with glycopeptide antibiotics. In this example, primers and probes were used to detect the vanA and vanB genes of Enterococcus faecium (ATCC 700221, Manassas Va.) and Enterococcus faecalis (ATCC 700802, Manassas Va.), also known as Vancomycin Resistant Enterococcus (VRE).

VRE was streaked onto blood agar media and incubated at 37° C. for 20 hours. Cell suspension was prepared from fresh growth by dilution in TE buffer (10 mM Tris HCl, 1 mM EDTA, pH 8.0) to a McFarland standard of 0.5, which equates to approximately 1×10⁸ colony forming units per milliliter (CFU/mL). One hundred microliters of this cell suspension was extracted and isolated with the MagNA Pure LC system using the MagNA Pure LC DNA Isolation Kit III (Bacteria, Fungi) kit (instrument and reagents obtained from Roche, Indianapolis, Ind.) per manufacturer's instructions.

Primers and probes were synthesized by Integrated DNA Technologies (Coralville, Iowa). The vanA probe sequence, 5′ ACTGCAGCCTGATTTGGTCCACCTCGCCA (SEQ ID NO:3), was dual labeled by 6-carboxy-4′,5′-dichloro-2′,7′-dimethoxyfluorescein (JOE) and BHQ (BLACK HOLE QUENCHER, Integrated DNA Technologies, Coralville, Iowa) at the 5′- and 3′-position, respectively. The vanB probe sequence, 5′ TCCCATGACCGCGCAGCCGACCTCA (SEQ ID NO:6), was dual labeled by 6-carboxyfluorescein (FAM) and BHQ at the 5′- and 3′-position, respectively. Primer and probe sequences are listed in Table 1.

TABLE 1
Primer and Probe Sequences Used in the Detection of vanA and vanB
Genes in VRE.

| Name | Sequence 5′-3′ | Organism | Gene | Notes |
|---|---|---|---|---|
| SEQ ID NO: 3 | JOE-ACTGCAGCCTGATTT | VRE | vanA | 5′ JOE Fluorophore, |
| | GGTCCACCTCGCCA-BHQ | | | 3′ BHQ Quencher |
| SEQ ID NO: 1 | TTGTGCGGTATTGGGAAACAGTG | VRE | vanA | |
| SEQ ID NO: 2 | TTTCCGGCTCGACTTCCTGATGAATA | VRE | vanA | |
| SEQ ID NO: 6 | FAM-TCCCATGACCGCGC | VRE | vanB | 5′ FAM Fluorophore, |
| | AGCCGACCTCA-BHQ | | | 3′ BHQ Quencher |
| SEQ ID NO: 4 | AGAAGCAGCAGGACAATATGATGGA | VRE | vanB | |
| SEQ ID NO: 5 | GATACCGTGGCTCAACCGGATTT | VRE | vanB | |

Each sample was subjected to real-time PCR amplification for the vanA and vanB genes using the following optimized concentrations of primers, probe and enzyme, and thermocycle protocol. PCR amplification was performed in a total volume of 10 μL containing 5 microliters (μL) of sample and 5 μL of the following mixture: two primers (0.5 μL of 10 micromolar (μM) of each), probe (1 μL of 2 μM), MgCl₂ (2 μL of 25 mM) and LightCycler® DNA Master Hybridization Probes (1 μL of 10×, Roche, Indianapolis, Ind.). Amplification was performed on the LightCycler® 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

Results were analyzed using the software provided with the Roche LightCycler® 2.0 Real Time PCR System. The primers successfully amplified the vanA and vanB genes under the conditions presented in this example as shown in Tables 2 and 3.

TABLE 2
Real-Time PCR Amplification of vanA From VRE (Enterococcus
faecium (ATCC 700221) DNA was purified using the MagNA
Pure System and serially diluted in TE buffer. Real time PCR
was performed in duplicate using 5 μL of each sample.
Real Time PCR Amplification of vanA From VRE

| Sample | Ct¹ | |
|---|---|---|
| 1 × 10⁸ CFU/mL E. faecium DNA | 12.01 | 11.65 |
| 1 × 10⁷ CFU/mL E. faecium DNA | 16.15 | 15.21 |
| 1 × 10⁶ CFU/mL E. faecium DNA | 19.42 | 19.89 |
| 1 × 10⁵ CFU/mL E. faecium DNA | 23.53 | 23.29 |
| 1 × 10⁴ CFU/mL E. faecium DNA | 26.36 | 26.76 |

¹Ct, cycle threshold.

These results show that the vanA gene was successfully amplified and detected using the primers and probes of SEQ ID NO:1, 2, and 3.

TABLE 3
Real-Time PCR Amplification of vanB From VRE (Enterococcus
faecalis (ATCC 700802) DNA was purified using the MagNA
Pure System, and serially diluted in TE buffer. Real time PCR
was performed in duplicate using 5 μL of each sample.
Real Time PCR Amplification of vanB From VRE

| Sample | Ct¹ | |
|---|---|---|
| 1 × 10⁸ CFU/mL E. faecalis DNA | 15.38 | 14.68 |
| 1 × 10⁷ CFU/mL E. faecalis DNA | 19.1 | 18.66 |
| 1 × 10⁶ CFU/mL E. faecalis DNA | 22.65 | 21.92 |
| 1 × 10⁵ CFU/mL E. faecalis DNA | 25.98 | 25.32 |
| 1 × 10⁴ CFU/mL E. faecalis DNA | 29.56 | 28.65 |

¹Ct, cycle threshold.

These results show that the vanB gene was successfully amplified and detected by SEQ ID 4-6.

Several reference strains of Enterococcus were tested to determine if their known vancomycin resistance profile correlated to the presence of the vanA or vanB genes in the bacterial chromosome. Specifically, Enterococcus isolates purchased from the American Type Culture Collection (ATCC) (Manassas, Va.) were streaked onto blood agar media and incubated at 37° C. for 20 hours. Cell suspension was prepared from fresh growth by dilution in TE buffer (10 mM Tris HCl, 1 mM EDTA, pH 8.0) to a McFarland standard of 0.5, which equates to approximately 1×10⁸ CFU/mL. One hundred microliters of this cell suspension was extracted and isolated with the MagNA Pure LC system using the MagNA Pure LC DNA Isolation Kit III (Bacteria, Fungi) kit (instrument and reagents obtained from Roche, Indianapolis, Ind.) per manufacturer's instructions.

Each sample was subjected to real-time PCR amplification for the vanA and vanB genes using the following optimized concentrations of primers, probe and enzyme, and thermocycle protocol. PCR amplification was performed in a total volume of 10 μL containing 5 μL of sample and 5 μL of the following mixture: two primers (0.5 μL of 10 μM of each), probe (1 μL of 2 μM), MgCl₂ (2 μL of 25 mM) and LightCycler® DNA Master Hybridization Probes (1 μL of 10×, Roche, Indianapolis, Ind.). Amplification was performed on the LightCycler® 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition). Results may be seen in Table 4.

TABLE 4
Presence of vanA and vanB Genes in Various Enterococcus Strains. Strains are displayed
using their ATCC designations. Resistances are listed as provided by ATCC. The column
Vancomycin Resistance Genotype indicates the presence of a vanA or vanB gene as
determined by real-time PCR amplification, or the absence of both (none).

| | | | Vancomycin |
|---|---|---|---|
| Description | Strain | Resistance | Resistance Genotype |
| ATCC 51559 | Enterococcus faecium; | ampicillin, ciprofloxacin, | VanA |
| | Strain MMC4 | gentamicin, rifampin, | |
| | | teicoplanin, vancomycin | |
| ATCC 51575 | Enterococcus faecalis | gentamicin, streptomycin, | VanB |
| | | vancomycin | |
| ATCC 700802 | Enterococcus faecalis | gentamicin, vancomycin, | VanB |
| | | and teicoplanin | |
| ATCC 700221 | Enterococcus faecium | vancomycin | VanA |
| ATCC 43076 | Enterococcus | N/A | None |
| | saccharolyticus | | |
| ATCC 11576 | Enterococcus durans | N/A | None |
| ATCC 29212 | Enterococcus faecalis | N/A | None |
| ATCC 14506 | Enterococcus faecalis | N/A | None |
| ATCC 49032 | Enterococcus faecium | N/A | None |
| ATCC 27270 | Enterococcus faecium | N/A | None |
| ATCC 49533 | Enterococcus faecalis | streptomycin | None |
| ATCC 7080 | Enterococcus faecalis | N/A | None |
| ATCC 19433 | Enterococcus faecalis | N/A | None |
| ATCC 49452 | Enterococcus faecalis | N/A | None |
| ATCC 49532 | Enterococcus faecalis | gentamicin | None |
| ATCC 33186 | Enterococcus faecalis | N/A | None |
| ATCC 51299 | Enterococcus faecalis | vancomycin (low-level | VanB |
| | | resistance) | |
| ATCC 35667 | Enterococcus faecium | N/A | None |
| ATCC 6569 | Enterococcus faecium | N/A | None |

These results suggest that SEQ ID 1-6 are specific to vanA and vanB genes within the enterococcal chromosome, and do not hybridize to non-vanA or vanB sequences.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. (canceled)

2. A method for detecting a drug-resistant microbe in a biological sample comprising:
amplifying a target polynucleotide present in a biological sample to result in an amplified product, wherein the biological sample is contacted with a first vanA primer and a second vanA primer under suitable conditions to result in an amplified product, wherein the first primer comprises a nucleotide sequence with at least about 80% identity to SEQ ID NO:1, and the second primer comprises a nucleotide sequence with at least about 80% identity to SEQ ID NO:2, wherein the primer pair amplifies nucleotides 648-751 of SEQ ID NO:7; and
detecting the amplified product, wherein the presence of the amplified product is indicative of the presence of a drug-resistant microbe in the biological sample.

3. A method for detecting a drug-resistant microbe in a biological sample comprising:
amplifying a target polynucleotide present in a biological sample to result in an amplified product, wherein the biological sample is contacted with a first vanB primer and a second vanB primer under suitable conditions to result in an amplified product, wherein the first primer comprises a nucleotide sequence with at least about 80% identity to SEQ ID NO:4, and the second primer comprises a nucleotide sequence with at least about 80% identity to SEQ ID NO:5, wherein the primer pair amplifies nucleotides 492-630 of SEQ ID NO:8; and
detecting the amplified product, wherein the presence of the amplified product is indicative of the presence of a drug-resistant microbe in the biological sample.

4. (canceled)

5. A method for detecting the absence of a drug-resistant microbe in a biological sample comprising:
contacting a biological sample with a first vanA primer and a second vanA primer to form a mixture, wherein the first primer comprises a nucleotide sequence with at least about 80% identity to SEQ ID NO:1, and the second primer comprises a nucleotide sequence with at least about 80% identity to SEQ ID NO:2, wherein the primer pair amplifies nucleotides 648-751 of SEQ ID NO:7;
exposing the mixture to conditions suitable to form an amplified product if a vanA polynucleotide is present in the biological sample; and
detecting the absence of the amplified product, wherein the absence of the amplified product is indicative of the absence of a drug-resistant microbe in the biological sample.

6. A method for detecting the absence of a drug-resistant microbe in a biological sample comprising:
contacting a biological sample with a first vanB primer and a second vanB primer to form a mixture, wherein the first primer comprises a nucleotide sequence with at least about 80% identity to SEQ ID NO:4, and the second primer comprises a nucleotide sequence with at least about 80% identity to SEQ ID NO:5, wherein the primer pair amplifies nucleotides 492-630 of SEQ ID NO:8;
exposing the mixture to conditions suitable to form an amplified product if a vanA polynucleotide is present in the biological sample; and
detecting the absence of the amplified product, wherein the absence of the amplified product is indicative of the absence of a drug-resistant microbe in the biological sample.

7. The method of claim 2 wherein the microbe is a member of the genus Enterococcus.

8. The method of claim 7 wherein the member of the genus Enterococcus is E. faecalis.

9. (canceled)

10. The method of claim 2, wherein the target polynucleotide is a vanA polynucleotide, wherein the first primer comprises a nucleotide sequence with at least about 80% identity to SEQ ID NO:1, and the second primer comprises a nucleotide sequence with at least about 80% identity to SEQ ID NO:2, wherein the primer pair amplifies nucleotides 648-751 of SEQ ID NO:7.

11. The method of claim 10 wherein the first primer comprises SEQ ID NO:1 and the second primer comprises SEQ ID NO:2.

12. The method of claim 2, wherein the target polynucleotide is a vanA polynucleotide, wherein the probe comprises a nucleotide sequence with at least about 80% identity to SEQ ID NO:3 and hybridizes to SEQ ID NO:7.

13. (canceled)

14. The method of claim 2, wherein the target polynucleotide is a vanB polynucleotide, wherein the first primer comprises a nucleotide sequence with at least about 80% identity to SEQ ID NO:4, and the second primer comprises a nucleotide sequence with at least about 80% identity to SEQ ID NO:5, wherein the primer pair amplifies nucleotides 492-630 of SEQ ID NO:8.

15. The method of claim 14 wherein the first primer comprises SEQ ID NO:4 and the second primer comprises SEQ ID NO:5.

16. The method of claim 2, wherein the target polynucleotide is a vanB polynucleotide, wherein the probe comprises a nucleotide sequence with at least about 80% identity to SEQ ID NO:6 and hybridizes to SEQ ID NO:8.

17-18. (canceled)

19. The method of claim 2 wherein the biological sample is from an individual suspected of infection with a drug-resistant microbe.

20. The method of claim 19 wherein the biological sample comprises fecal material.

21. The method of claim 2 further comprising obtaining the biological sample.

22. The method of claim 2 wherein the detecting is performed after each cycling step.

23. The method of claim 2 wherein the first vanA primer comprises SEQ ID NO:1 and the second vanA primer comprises SEQ ID NO:2.

24. The method of claim 3 wherein the first vanB primer comprises SEQ ID NO:4 and the second vanB primer comprises SEQ ID NO:5.

25. The method of claim 2 wherein the amplifying further comprises contacting the biological sample with a probe, wherein the T_{M} of the probe is at least 8° C. higher than the T_{M} of the first primer and the second primer.

26. The method of claim 5 wherein the amplifying further comprises contacting the biological sample with a probe to form a mixture comprising the first vanA primer, the second vanA primer, and the probe, wherein the T_{M} of the probe is at least 8° C. higher than the T_{M} of the first primer and the second primer

27. The method of claim 6 wherein the amplifying further comprises contacting the biological sample with a probe to form a mixture comprising the first vanB primer, the second vanB primer, and the probe, wherein the T_{M} of the probe is at least 8° C. higher than the T_{M} of the first primer and the second primer

28. The method of claim 25, wherein the probe comprises a fluorophore and a quencher.

29. The method of claim 28 wherein the detecting comprises detecting a fluorophore.

30. The method of claim 25, wherein the amplifying comprises a DNA polymerase comprising 5′ to 3′ exonuclease activity.

31. A method for isolating a polynucleotide comprising:
providing a mixture comprising single stranded polynucleotides;
exposing the mixture to an oligonucleotide under conditions suitable for specific hybridization of the oligonucleotide to a single stranded polynucleotide to result in a hybrid, wherein the oligonucleotide comprises a nucleotide sequence selected from at least about 80% identity to SEQ ID NO:1, at least about 80% identity to SEQ ID NO:2, at least about 80% identity to SEQ ID NO:3, at least about 80% identity to SEQ ID NO:4, at least about 80% identity to SEQ ID NO:5, or at least about 80% identity to SEQ ID NO:6, and wherein the oligonucleotide comprises an affinity label; and
washing the hybrid.

32. The method of claim 31 further comprising attaching the oligonucleotide to a solid phase material after the exposing.

33. The method of claim 31 wherein the oligonucleotide is attached to a solid phase material before the exposing.

34. The method of claim 31 wherein the mixture is obtained from a biological sample.

35. The method of claim 34 wherein the biological sample comprises fecal material.

36. A kit comprising packaging materials, a first vanA primer, a second vanA primer, and a probe, and wherein the T_{M} of the probe is at least 8° C. higher than the T_{M} of the first and second vanA primers, wherein the probe comprises a nucleotide sequence with at least about 80% identity to SEQ ID NO:3 and hybridizes to SEQ ID NO:7.

37. (canceled)

38. A kit comprising packaging materials, a first vanB primer, a second vanB primer, and a probe, and wherein the T_{M} of the probe is at least 8° C. higher than the T_{M} of the first and second vanB primers, wherein the probe comprises a nucleotide sequence with at least about 80% identity to SEQ ID NO:6 and hybridizes to SEQ ID NO:8.

39. (canceled)

40. The kit of claim 36 wherein the probe comprises a fluorophore and a quencher.

41. The kit of claim 36 wherein the first primer comprises SEQ ID NO:1 and the second primer comprises SEQ ID NO:2.

42. The kit of claim 38 wherein the first primer comprises SEQ ID NO:4 and the second primer comprises SEQ ID NO:5.

43. A kit comprising packaging materials, a first vanA primer and a second vanA primer, wherein the first primer comprises a nucleotide sequence with at least about 80% identity to SEQ ID NO:1, and the second primer comprises a nucleotide sequence with at least about 80% identity to SEQ ID NO:2, wherein the primer pair amplifies nucleotides 648-751 of SEQ ID NO:7.

44. The kit of claim 43 wherein the first primer comprises SEQ ID NO:1 and the second primer comprises SEQ ID NO:2.

45. A kit comprising packaging materials, a first vanB primer and a second vanB primer, wherein the first primer comprises a nucleotide sequence with at least about 80% identity to SEQ ID NO:4, and the second primer comprises a nucleotide sequence with at least about 80% identity to SEQ ID NO:5, wherein the primer pair amplifies nucleotides 492-630 of SEQ ID NO:8.

46. The kit of claim 45 wherein the first primer comprises SEQ ID NO:4 and the second primer comprises SEQ ID NO:5.

47. An isolated polynucleotide comprising a nucleotide sequence with at least about 80% identity to SEQ ID NO:1, wherein the polynucleotide amplifies a polynucleotide comprising nucleotides 648-751 of SEQ ID NO:7 when used with SEQ ID NO:2.

48. An isolated polynucleotide comprising a nucleotide sequence with at least about 80% identity to SEQ ID NO:2, wherein the polynucleotide amplifies a polynucleotide comprising nucleotides 648-751 of SEQ ID NO:7 when used with SEQ ID NO:1.

49. An isolated polynucleotide comprising a nucleotide sequence with at least about 80% identity to SEQ ID NO:4, wherein the polynucleotide amplifies a polynucleotide comprising nucleotides 492-630 of SEQ ID NO:8 when used with SEQ ID NO:5 to result in an amplified product of about 139 nucleotides.

50. An isolated polynucleotide comprising a nucleotide sequence with at least about 80% identity to SEQ ID NO:5, wherein the polynucleotide amplifies a polynucleotide comprising nucleotides 492-630 of SEQ ID NO:8 when used with SEQ ID NO:4.


SEQUENCE LISTING (8 sequences)

SEQ ID NO: 1 - DNA, 23 bp, artificial
  primer sequence
ttgtgcggtattgggaaacagtg

SEQ ID NO: 2 - DNA, 26 bp, artificial
  primer sequence
tttccggctcgacttcctgatgaata

SEQ ID NO: 3 - DNA, 29 bp, artificial
  primer sequence
actgcagcctgatttggtccacctcgcca

SEQ ID NO: 4 - DNA, 25 bp, artificial
  primer sequence
agaagcagcaggacaatatgatgga

SEQ ID NO: 5 - DNA, 23 bp, artificial
  primer sequence
gataccgtggctcaaccggattt

SEQ ID NO: 6 - DNA, 25 bp, artificial
  primer sequence
tcccatgaccgcgcagccgacctca

SEQ ID NO: 7 - DNA, 1032 bp, Enterococcus faecalis
atgaatagaataaaagttgcaatactgtttgggggttgctcagaggagcatgacgtatcggtaaaatctgcaatagagatagccgctaacattaataaagaaaaatacgagccgttatacattggaattacgaaatctggtgtatggaaaatgtgcgaaaaaccttgcgcggaatgggaaaacgacaattgctattcagctgtactctcgccggataaaaaaatgcacggattacttgttaaaaagaaccatgaatatgaaatcaaccatgttgatgtagcattttcagctttgcatggcaagtcaggtgaagatggatccatacaaggtctgtttgaattgtccggtatcccttttgtaggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagtgccgcgttagttgttggcgaggtggaccaaatcaggctgcagtacggaatctttcgtattcatcaggaagtcgagccggaaaaaggctctgaaaacgcagttataaccgttcccgcagacctttcagcagaggagcgaggacggatacaggaaacggcaaaaaaaatatataaagcgctcggctgtagaggtctagcccgtgtggatatgtttttacaagataacggccgcattgtactgaacgaagtcaatactctgcccggtttcacgtcatacagtcgttatccccgtatgatggccgctgcaggtattgcacttcccgaactgattgaccgcttgatcgtattagcgttaaaggggtga

SEQ ID NO: 8 - DNA, 732 bp, Enterococcus faecalis
aacattaatactgaaaaattcgatccgcactacatcggaattacaaaaaacggcgtatggaagctatgcaagaagccatgtacggaatgggaagccgatagtctccccgccatattctccccggataggaaaacgcatggtctgcttgtcatgaaagaaagagaatacgaaactcggcgtattgacgtggctttcccggttttgcatggcaaatgcggggaggatggtgcgatacagggtctgtttgaattgtctggtatcccctatgtaggctgcgatattcaaagctccgcagcttgcatggacaaatcactggcctacattcttacaaaaaatgcgggcatcgccgtccccgaatttcaaatgattgaaaaaggtgacaaaccggaggcgaggacgcttacctaccctgtctttgtgaagccggcacggtcaggttcgtcctttggcgtaaccaaagtaaacagtacggaagaactaaacgctgcgatagaagcagcaggacaatatgatggaaaaatcttaattgagcaagcgatttcgggctgtgaggtcggctgcgcggtcatgggaaacgaggatgatttgattgtcggcgaagtggatcaaatccggttgagccacggtatcttccgcatccatcaggaaaacgagccggaaaaaggctcagagaatgcgatgattatcgttccagcagacattccggtcgaggaacgaaatcgggtgcaagaa